(19) United States
(12) Patent Application Publication
(10) Pub. No.: US 2003/0049797 A1
(43) Pub. Date: Mar. 13, 2003

(54) Hybrid proteins for autoimmune disease

(76) Inventors: Yoshikazu Yuki, Kobe-shi (JP); Shigezo Udaka, Ashiya-shi (JP)

Correspondence Address: PILLSURY WINTHROP LLP, INTELLECTUAL PROPERTY GROUP, 11682 EL CAMINO REAL, SUITE 200, SAN DIEGO, CA, 94105, US

(21) Appl. No.: 09/836,433

(22) Filed: Apr. 16, 2001

(51) Int. Cl. C12P 21/04; C07H 21/04; A61K 38/46; C12N 9/64

(52) U.S. Cl. 435/69.7; 435/226; 536/23.2; 424/94.6

(57) ABSTRACT

Autoantigen-tolerogen fusion polypeptides, polynucleotides, expression vectors and host cells useful in inducing tolerance to autoantigens are provided. Preferred autoantigen fusion polypeptides contain a peptide encompassing proteolipid protein amino acids 139-151 fused to cholera toxin B-subunit. A Bacillus brevis expression-secretion system and methods for making autoantigen fusion polypeptides are also disclosed. The invention also includes methods for inducing tolerance to autoantigens, as well as treating and ameliorating the symptoms of neurodegenerative disease.

### FIELD OF THE INVENTION

The present invention generally relates to autoantigen-tolerogen fusion polypeptides useful for treatment of autoimmune disease, polynucleotides encoding fusion polypeptides, expression vectors and methods of producing autoantigen fusion polypeptides, particularly expression in Bacillus brevis. Also provided are methods of using autoantigen fusion polypeptides for inducing tolerance to autoantigens and treating neurodegenerative disease.

### BACKGROUND

An estimated four percent of the population is currently affected by autoimmune diseases including forms of multiple sclerosis, diabetes, arthritis and lupus. Autoimmunity results when the cells of the immune system recognize and attack so called “self” antigens or autoantigens that are normally present in and indeed produced by the body itself. As immune responses are in general destructive, i.e. meant to destroy invasive foreign antigens, autoimmune responses can cause destruction of the body's own tissue.

Steroid treatment is the most common therapy for autoimmune disease. However, steroids non-specifically repress a wide variety of both desirable and undesirable immune functions, may be only partially effective, and are associated with significant adverse physiological and psychological side effects.

An alternative to steroid treatment of autoimmune disease is therapeutic induction of tolerance to autoantigen targets. Tolerance is the mechanism animals normally use to avoid recognizing and thereby attacking autoantigens. In experimental animal models, high doses of autoantigens administered mucosally have been found to effective for prophylaxis of cellular autoimmune responses. However, such treatments are of limited efficacy in pre-sensitized animals or patients with existing autoimmune disease.

Promising studies indicate that autoantigenic peptides chemically conjugated to “tolerogens,” such as cholera toxin B-subunit (CTB), may be effective in both prophylaxis and treatment of pre-existing autoimmune. Nevertheless, heterogeneity, limited commercial availability and potential toxicity of chemical autoantigen-tolerogen conjugates limit their widespread use as tolerance-inducing therapeutics.

### SUMMARY OF THE INVENTION

The present invention provides an isolated polynucleotide encoding a fusion polypeptide comprising at least one proteolipid protein (PLP) fragment fused in frame to a tolerogen polypeptide. In one embodiment, this invention relates to polynucleotides encoding amino acids 139-151 of proteolipid protein, naturally occurring autoantigenic variants thereof, and synthetic variants capable of inducing tolerance to proteolipid protein autoantigens.

According to the present invention, the polynucleotide may encode PLP fragments fused to the cholera toxin B (CTB) subunit or CTB variants capable of functioning as a tolerogen when fused to an autoantigen peptide. Encoded fusion polypeptides of the invention may optionally contain a flexible hinge region between the autoantigen and tolerogen peptides.

Also included in the invention are expression vectors comprising fusion polypeptide encoding polynucleotide sequences, host cells contain such vectors and fusion polypeptides expressed therefrom. Preferably, the host is a Bacillus brevis.

Methods according to the invention provide producing an autoantigen fusion protein comprising the steps of: 1) providing an expression vector, wherein a first polynucleotide sequence encoding an autoantigen is fused in-frame to a second polynucleotide sequence encoding a tolerogen, wherein the resulting fused polynucleotide sequences are operatively associated with regulatory sequences; 2) expressing autoantigen fusion polypeptide from the expression vector in Bacillus host cells; and 3) recovering the autoantigen fusion polypeptide.

Also provided is a method of treating a neurodegenerative disease comprising the steps of: 1) providing an expression vector, wherein a first polynucleotide sequence encoding an autoantigen is fused in-frame to a second polynucleotide sequence encoding a tolerogen, wherein the resulting fused polynucleotide sequences are operatively associated with regulatory sequences; 2) expressing autoantigen fusion polypeptide from the expression vector in Bacillus host cells; 3) recovering the autoantigen fusion polypeptide; and 4) administering the autoantigen fusion polypeptide to a patient.

Preferably, the expression vector encodes a PLP-CTB autoantigen fusion polypeptide produced in Bacillus brevis cells. Alternatively, the expression vector encodes a myelin oligodendrocyte glycoprotein-(MOG)tolerogen autoantigen fusion polypeptide and may contain a flexible hinge polypeptide.

The autoantigen fusion polypeptide may be useful for treatment of multiple sclerosis or for ameliorating symptoms of neurodegenerative disease such as loss of mobility, spasticity, pain, tremor, abnormal eye movements, paroxysmal symptoms, paralysis, bladder and bowel dysfunction, sexual disturbances, fatigue and depression.

Preferably, autoantigen fusion polypeptides of the invention are administered mucosally, for example, orally or nasally.

Additionally, a method of inducing tolerance to an autoantigen is provided comprising administering an effective amount of pharmaceutical composition containing autoantigen fusion polypeptides and a pharmaceutically acceptable carrier.

These and other aspects of the present invention should be apparent to those skilled in the art from the teachings herein.

### BRIEF DESCRIPTION OF THE DRAWINGS

FIG. 1 shows CTB expression-secretion vector pNU212-CTB. The promoter and signal peptide of the MWP (middle wall protein) gene are represented by the hatched bar and the sequence coding for CTB by the filled bar. Arrows indicate the direction of transcription. “Ori” signifies “Origin of replication”. “Em^{r}” is the erythromycin resistance gene. The nucleotide [SEQ ID NO.:27] and amino acid [SEQ ID NO.:28] sequences around the signal peptide cleavage site (arrow) of the fused gene are shown at the bottom.

FIG. 2 shows construction of CTB-MBP 84-102 hybrid protein expression-secretion vector'pNU212-CTB-MBPp. The promoter and signal peptide of the MWP gene are represented by the hatched bar and the sequence coding for CTB-MBP 84-102, by the filled bar. Arrows indicate the direction of transcription. “Ori” signifies “Origin of replication”. The nucleotide [SEQ ID NO.:29] and amino acid [SEQ ID NO.:30] sequences around the linking site with MBP 84-102 (arrow) of the fused gene are shown at the bottom.

FIG. 3 shows construction of CTB-PLP 139-151(C140S) hybrid protein expression-secretion vector pNU212-CTB-PLPp. The promoter and signal peptide of the MWP gene are represented by the hatched bar and the sequence coding for CTB-PLP 139-151(C140S) by the filled bar. Arrows indicate the direction of transcription. “Ori” signifies “Origin of replication”. The nucleotide [SEQ ID NO.:31] and amino acid [SEQ ID NO.:32] sequences around the linking site with PLP 139-151(C140S) (arrow) of the fused gene are shown at the bottom.

FIG. 4 shows the construction of CTB-PLP 139-151 (C140S) with a hinge peptide hybrid protein expression-secretion vectors. The promoter and signal peptide of the MWP gene are represented by the hatched bar aid the sequence coding for CTB-hinge-PLP peptide by the filled bar. Arrows indicate the direction of transcription. “Ori” signifies “Origin of replication”. The nucleotide [SEQ ID NOS.:37] and amino acid [SEQ ID NOS.:38] sequences around the linking site with these autoantigen peptides of the fused gene are shown at the bottom.

FIG. 5 shows construction of CTB-collagen Type II 255-270 hybrid protein expression-secretion vectors. The promoter and signal peptide of the MWP gene are represented by the hatched bar aid the sequence coding for CTB-collagen peptide by the filled bar. Arrows indicate the direction of transcription. “Ori” signifies “Origin of replication”. The nucleotide [SEQ ID NOS.:33 and 35] and amino acid [SEQ ID NOS.:34 and 36] sequences around the linking site with these autoantigen peptides of the fused gene are shown at the bottom.

FIG. 6 shows comparison of the affinities of the rCTB-PLP 139-151(C140S) and rCTB-MBP 84-102 hybrid proteins and the native form of rCTB for the GM1 receptor by competitive ELISA. Each protein was adjusted to an equimolar concentration. Serially diluted two-fold concentrations of individual samples were then mixed with a fixed amount of biotinylated CTB and reacted with GM1 bound on the solid phase. Data are the average of six measurements.

Definitions

“Antigen” refers to peptides, polypeptides and other species recognized by cells of the immune system, including B and T lymphocytes. “Autoantigens” or “self antigens” are molecules normally present in a host that are typically produced by the host itself. In “autoimmune” disease, the host recognizes autoantigens by producing an unwanted immune response to the autoantigen that results in destruction of the autoantigen molecule and/or surrounding tissue.

As used herein, immunological “tolerance” refers to a reduction in immunological reactivity of a host towards a specific antigen or antigens. The specific antigens comprise immune determinants that, in the absence of tolerance, cause an unwanted immune response, such as, for example, acute or chronic inflammation caused by delayed-type hypersensitivity (DTH). DTH is characterized by an immune response at the site of exposure to antigen, which comprises an initial infiltration of neutrophils followed by accumulation of T lymphocytes and blood monocytes, deposition of fibrin, and induration. Tolerance can be induced to both foreign antigens not normally present in the organism or to autoantigens produced by the organism itself.

“Tolerogen” as used herein, refers to a polypeptide or other species that, when incorporated into a tolerance-inducing composition, promotes the development of immune tolerance. Tolerogens include, for example, mucosa-binding polypeptides that facilitate site-specific delivery and presentation of the linked antigenic species, such as the autoantigen peptides of the present invention, to mucosal inductive sites.

### DETAILED DESCRIPTION OF THE INVENTION

The present invention provides an isolated polynucleotide encoding a fusion polypeptide comprising at least one proteolipid protein (PLP) fragment fused in frame to a tolerogen polypeptide. According to one embodiment, the PLP fragment comprises amino acids 139-151 of proteolipid protein (PLP; SEQ ID NO.:1 [amino acid sequence 139-151]).

According to the invention, the PLP fragment may be any peptide that is a target for autoimmune disease such as those described by Tuohy, Biochemical Res. 19:935-933 (1994). For example, the PLP fragment may be one selected from the group consisting of SEQ ID NO.:2; SEQ ID NO.:3; SEQ ID NO.:4; SEQ ID NO.:5; SEQ ID NO.:6; SEQ ID NO.:7; SEQ ID NO.:8; SEQ ID NO.:9; SEQ ID NO.:10; SEQ ID NO.:11; SEQ ID NO.:12; and SEQ ID 13.

Other embodiments include variants of PLP peptides, including naturally occurring variants of PLP, such as allelic variants, that may be present in individual members of a population and give rise to specific autoimmune responses in those individuals. The invention also contemplates that that tolerance may be induced to an autoantigen, such as PLP, through the use of synthetic variants that differ from the naturally occurring autoantigen by one or more amino acids. A nonlimiting example of synthetic PLP variants of the invention are those in which one or more cysteine residues in naturally occurring PLP sequence is substituted by serine residues for stability of the chimeric protein.

In one aspect of the invention the C-terminal amino acid of a PLP peptide is fused to the N-terminal amino acid of one or more additional PLP peptides in a head to tail manner. A hybrid protein comprised of PLP multiple peptides according to the invention can contain a variable number of peptides units. Preferably, 1-3 PLP peptides are fused in a head to tail manner to the tolerogen.

According to the invention, the encoded fusion protein also comprises a tolerogen polypeptide which may be any polypeptide, or fragment thereof, capable of promoting tolerance to an associated autoantigen. In one embodiment, the tolerogen polypeptide is cholera toxin B subunit (CTB) [SEQ ID NO.:14 [amino acid sequence CTB]). In another embodiment, the tolerogen polypeptide is a variant of cholera toxin B subunit that is capable of functioning as a tolerogen when fused to an autoantigen peptide. An autoantigen peptide, such as PLP, can be fused to either the N-terminus or C-terminus of CTB. Preferably, the autoantigen peptide is fused to the C-terminus of CTB as it has been suggested that the N-terminus of CTB interacts with GM-1-ganglioside when functioning in a tolerogen capacity. (Zhang, et aL (1995) J. Mol. Biol. 251: 550-562). In one aspect of the invention, CTB-autoantigen fusion proteins are capable of forming pentameric structures capable of binding GM-1.

In one embodiment of the invention, the fusion polypeptide also contains a flexible hinge polypeptide between the autoantigen amino acids and the tolerogen polypeptide. Preferably, the hinge polypeptide consists of 2-8 amino acids such as GP or GPG or PGPG or DPVDPGS or LGGPVDP (Lipscombe, et al. (1991) Mol. Mircobiol. 5: 1385-1392; Jagusztyn-Krynicka, et al. (1993) Infect. Immunol. 61: 1004-1015). The flexible hinge polypeptide may, for example, comprise the sequence set forth in SEQ ID NOS:15-18. Preferable, are polypeptides that are flexible in that they are able to bend or twist at the hinge region, such as those containing proline.

According to one embodiment, polynucleotides of the present invention encode, for example, the polypeptides set forth in SEQ ID NOS.20 and 22. Nonlimiting examples of polynucleotides of the invention include the sequences set forth in SEQ ID NOS:21 and 22.

The present invention also provides expression vectors that direct the synthesis of fusion proteins of the invention, such as expression vectors comprising the polynucleotide of the invention of operatively linked to at least one transcriptional regulatory element. Also included in the invention are host cells containing these expression vectors.

Any transcriptional regulatory elements functional in the desired host may be used. For example, the regulatory elements may include promoters, operators and enhancers. Also contemplated are inducible regulatory elements, such at those responsive to nutrients, hormones, antibiotics and the like. The invention also contemplates that additional regulatory transcriptional, translation or processing elements, such as polyadenylation sites, CAP sites, splice junctions, and signal sequences may be incorporated into the polynucleotide to increase, for example, stability of transcribed RNA or processing of translated peptides. Particularly preferred are promoters functional in B. brevis, including those derived from strains of B. brevis. For example, in one embodiment, a promoter of a major extracellular protein gene of B. brevis 47 (FERM-7224) or B. brevis HPD31 (FERM BP-1087) can be used. In such embodiments, it is essential that the polynucleotide containing a promoter region further contains an SD sequence, a translation initiation codon in addition to a part of the major extracellular protein gene.

To operationally link polynucleotides encoding tolerogen-autoantigen peptides to the transcriptional regulatory regions, the polynucleotide may be ligated to 3′ terminal of polynucleotide sequences excised from the chromosome of B. brevis. Autoantigen fusion polypeptides of the invention, such as CTB-PLP fusions, can be recovered from either intracellularly or extracellularly expressed constructs. However, if extracellular accumulation is desired, a region coding for a signal peptide must be included in the polynucleotide construct, upstream from autoantigen fusion polypeptide sequences. A signal peptide of a major extracellular protein of B. brevis 47 or B. brevis HPD31 may be used, such as the signal peptide is of the MWP (Middle Wall Protein) of B. brevis 47.

Methods for fusing polynucleotides encoding various polypeptides “in frame” such that they encode each polypeptide region in the same translational reading frame are well known in the art including polymerase chain reaction and restriction endonuclease digestion-ligation methods. It will be appreciated that expression of fused peptide sequences provides a consistent and homogeneous autoantigen fusion polypeptide as compared to chemically coupled synthetic or expressed conjugates. Methods for constructing expression plasmids, such as those containing autoantigen fusion polypeptide sequences as described, are well known, as for example, described in Sambrook, et al., Molecular Cloning, A Laboratory Manual (Cold Spring Habor Laboratory (1990)). A preferred expression vector for CTB-autoantigen peptide expression (pNU) is described below in the examples.

It will be appreciated that expression vector constructs of the invention may be expressed in a variety of host cell types including bacteria, yeast, insect cell, plant and animal cells. The host may be any cell which does not provide a strong negative influence on autoantigen-tolerogen fusion polypeptides, such as PLP-CTB fusions, produced by the expression plasmids, and may be B. brevis 47, B. brevis HPD31 or the like. A preferred host is B. brevis 31-OK (FERM BP-4573) obtained from B. brevis HPD31 by mutagenesis. Since this mutant substantially does not exhibit protease activity toward CTB-autoantigen fusion polypeptides, it can stably support expression of fusion peptides produced and accumulated in culture.

The use of B. brevis as a recombinant hybrid protein expression system has also been shown to offer several other advantageous properties. For example, the B. brevis system produces high levels of recombinant proteins in culture medium while posing little threat of contamination by lipopolysaccharide (Inoue, et al. (1997) Appl. Microbiol. Biotechnol. 48: 487-489).

Methods for introduction of expression plasmids into host cells such as B. brevis are well known. For example, plasmids may be introduced by electroporation (Okamoto, et. al. (1997) Biosci. Biotech. Biochem. 61: 202-203). Transformed B. brevis cells may be cultured in nutrient medium to produce a large quantities of CTB-autoantigen fusion polypeptide, a major portion of which is extracellularly secreted. When B. brevis 31-OK is used as a host, CTB-autoantigen fusion polypeptides that are extracellularly secreted are also stably maintained. Thus, the CTB-autoantigen peptide can be efficiently recovered from the culture medium using B. brevis 31-OK host cells.

Secreted recombinant polypeptides produced in B. brevis have been shown to form correctly folded structures with appropriate biological activity (Yamagata, et al. (1989) Proc. Natl. Acad. Sci. USA 86: 3589-3593). Furthermore, the B. brevis PLP-CTB fusion protein is secreted as pentamer. Nontoxic CTB functions as a tolerogen based on its affinity for cell surface receptor GM-1-gangliosides expressed by cells in mucosal inductive sites such as the gut-associated lymphoreticular tissues (GALT). The pentameric structure of the CTB fusion protein not only facilitates site-specific delivery and presentation of the linked proteins to mucosal inductive sites but also increases the molar concentration of the antigen per molecule of CTB pentamer. Thus, production of CTB-autoantigen fusion polypeptides in B. brevis facilitates the pentameric structure required for binding to GM-1 as well as high levels of fusion polypeptide production.

In addition, B. brevis is considered a safe microorganism for production of recombinant proteins that will be administered to an animal or human because this bacteria is known to be a harmless resident of soil, milk, and cheese (Udaka and Yamagata (1993) Meth. Enzymol. 217: 23-33). Further, a major advantage of B. brevis, is the very low level of extracellular protease activity which promotes stability of secreted recombinant proteins (Yamagata, et al. (1989) Proc. Natl. Acad. Sci. USA 86: 3589-3593). These unique characteristics of the B. brevis expression system result in production of uniform autoantigen fusion polypeptides.

Methods for growing recombinant B. brevis and producing recombinant proteins are well know. For example, a nutrient medium used for culturing may contain a carbon source, nitrogen source, and if necessary inorganic salts. In addition, culturing can be carried out using a synthetic medium comprised mainly of a sugar and inorganic salts. When an auxotropic strain is used, a corresponding nutrient factor necessary for the growth is preferably added to the medium. If necessary, an antibiotic or antifoam is added to the medium. Cultures are grown in medium at an initial pH of 5.0 to 9.0, preferably 6.5 to 7.5. Culturing temperature is usually 15° C. to 42° C., and preferably 24° C. to 37° C., and culturing time is usually 16 to 360 hours, preferably 24 to 144 hours.

To recover autoantigen peptides from the culture, supernatant and microbial cells may be separated for example, by centrifugation or filtration. Conventional procedure for purification of protein, for example, salting out and various chromatography steps such as ion exchange chromatography and gel filtration chromatography or the like may be used to purify hybrid proteins. CTB-autoantigen peptide purification may include galactose immobilized gel chromatography steps (Uesaka, et al. (1994) Microb. Pathogen 16: 71-76). Preferably the amount of CTB-autoantigen peptides purified according to the method is 50 to 500 mg per 1 liter of culture media.

The present invention also encompasses fusion proteins expressed from the expression vectors of the invention, as well as pharmaceutical compositions comprising these fusion proteins and a pharmaceutically acceptable carrier. Preferably, fusion proteins of the invention are produced in B. brevis host cells.

CTB-autoantigen fusion polypeptides can be quantitated by amino acid analysis using a amino acid analyzer after hydrolysis. Amino acid sequencing, a cross-linking study and GM1 receptor binding assay showed this expression system produced uniformed recombinant CTB-autoantigen proteins with pentamer formation as well as GM1-binding activity.

The present invention also provides a method for producing an autoantigen fusion protein comprising the steps of: 1) providing an expression vector, wherein a first polynucleotide sequence encoding an autoantigen is fused in-frame to a second polynucleotide sequence encoding a tolerogen, wherein the resulting fused polynucleotide sequences are operatively associated with regulatory sequences; 2) expressing autoantigen fusion protein from said expression vector in Bacillus host cells; 3) recovering said autoantigen fusion protein. 4) administering said autoantigen fusion protein to a patient.

Autoantigen fusion polypeptides produced by methods of the invention may useful, for example, in the treatment of autoimmune disease, such as neurodegenerative autoimmune disease. Autoantigens contemplated by the invention include but are not limited to those associated with suppression of autoimmune diseases, preferably T-cell mediated auto immune diseases such as multiple sclerosis, insulin-dependent type 1 diabetes mellitus and rheumatoid arthritis. Autoantigens associated with suppression of autoimmune diseases or T-cell mediated autoimmune diseases include but are not limited to insulin, glutamate decarboxylase 65 (GAD65), heat shock protein 60 (HSP60), myelin basic protein (MBP), myelin oligodendrocyte protein (MOG), proteolipid protein (PLP), collagen type II (Tisch and McDevitt, (1996) Cell 85: 291-297; Steinman (1996) Cell 85: 299-302; Feldmann, et al. (1996) Cell 85: 307-310). Specific autoantigens peptides associated with suppression of T-cell mediated autoimmune diseases include but are not limited to insulin B chain 9-23 (Daniel and Wegmann (1996) Proc. Natl. Acad. Sci. USA 93: 956-960), GAD65 251-265, 521-535 (Tian, etal. (1996)J. Exp. Med. 183: 1561-1567), HSP60 443-457 (Elias, et al (1991)Proc. Natl. Acad. Sci. USA 88: 3088-3091), MBP 84-102 (Wucherpfennig and Hafler (1995)Ann. NY Acad. Sci. 756: 241-258), MOG 35-55 (Wilenborg, et al. (1996)J. Immunol. 157:3223-3227), PLP 139-151 (Kuchroo, et al. (1991)Pathobiology 59: 305-312), collagen type II 250-270 (Khare, et al. (1995) J. Immunol. 155: 3653-3659).

Thus, the invention also includes methods for the treatment of neurodegenerative disease comprising the steps of: 1) providing an expression vector, wherein a first polynucleotide sequence encoding an autoantigen is fused in-frame to a second polynucleotide sequence encoding a tolerogen, wherein the resulting fused polynucleotide sequences are operatively associated with regulatory sequences; 2) expressing autoantigen fusion polypeptide from the expression vector in Bacillus host cells; 3) recovering the autoantigen fusion polypeptide; and 4) administering the autoantigen fusion polypeptide to a patient.

The tolerogen polypeptide may, for example be any polypeptide that when fused to an autoantigen peptide, is capable of promoting tolerance to the associated antigen. A variety of tolerance-inducing polypeptides are know in the art. The skilled artisan will recognize that polypeptides that are capable of inducing tolerance when chemically coupled to autoantigens may also be suitable for constructing the fusion polypeptides of the invention. In one embodiment, the tolerogen polypeptide is cholera toxin B subunit as set forth in SEQ ID NO.:14, or variants thereof capable of functioning as a tolerogen when fused to an autoantigen peptide. The expression vector may further comprise a third polynucleotide sequence encoding a flexible hinge polypeptide located between said first and second polynucleotides.

The present invention also provides autoantigen fusion proteins produced according to the methods described above which may be useful for treating autoimmune disease. The autoantigen fusion proteins may be administered mucosally, preferably orally or nasally. It was recently shown that oral administration of the nontoxic cholera-toxin-B-subunit-(CTB-) conjugated chemically to myelin basic protein, insulin and collagen prevented EAE in Lewis rats (Sun, et al. (1996) Proc. Natl. Acad. Sci. USA 91: 10795-10799); spontaneous autoimmune diabetes in NOD mice (Bergerot, et al. (1997) Proc. Natl. Acad. Sci. USA 94: 4610-4614); and arthritis model in DBA/I mice (Tarkowski, et al (1999) Arthritis & Rheumatism 42: 1628-1634), respectively. Most importantly, the oral administration of CTB-conjugated autoantigen suppressed symptoms of these experimental autoimmune diseases even after disease induction in these animals. The administration to experimental mammals via nasal or oral route of CTB-autoantigen fusion polypeptides produces mucosally induced tolerance to the expressed mammalian autoantigen. The administration via nasal or oral route of the resulting CTB-autoantigen peptides may also suppress or prevent the development of the autoimmune diseases or T-cell mediated autoimmune diseases in mammals.

Also provided in the present invention are methods of ameliorating the symptoms of neurodegenerative disease comprising administering a therapeutically effective amount of an autoantigen fusion protein. In one embodiment, the autoantigen component of the autoantigen fusion protein comprises a PLP fragment, particularly amino acids 139-151. In another embodiment, the autoantigen peptide is a fragment of myelin oligodendrocyte glycoprotein, such as those comprising SEQ ID NOS.:24-26. In other embodiments, the autoantigen is a naturally occurring or synthetic variants of PLP or MOG. PLP, particularly the region encompassing amino acids 139-151, has been implicated as an autoantigen recognized in autoimmune multiple sclerosis. Similarly, fragments of MOG have been identified as targets for multiple sclerosis(de Rosbo, et al., Eur. J. Immunol. 27:3059-69 (1997); Kroepfl, etal., J. Neurochem 67:2219 (1996)). The methods of the invention may therefore be useful for treating neurodegenerative disease, particularly the autoimmune disease, multiple sclerosis. Symptoms of multiple sclerosis include: loss of mobility, spasticity, pain, tremor, abnormal eye movements, paroxysmal symptoms, paralysis, bladder and bowel dysfunction, sexual disturbances, fatigue and depression. Thus, it is contemplated that the methods of the present invention may be useful in reducing or ameliorating one or more of these symptoms in patients with autoimmune neurodegenerative disorders including multiple sclerosis.

Autoantigen fusions polypeptides of the present invention may be administered according to dosage regimens established in the art whenever specific pharmacological modification of the autoimmunity is required, for example, when it is desirable to induce tolerance to the autoantigen.

The present invention also provides pharmaceutical compositions comprising one or more autoantigen fusion polypeptides of the invention together with a pharmaceutically acceptable carrier, diluent or excipient. In one embodiment, effective amounts of the pharmaceutical compositions of the invention are administered as a method of inducing tolerance to an autoantigen.

Preferably compositions of the invention are in unit dosage forms such as powders, granules, aerosol or liquid sprays, drops, ampoules, or suppositories; for oral, intranasal, sublingual or rectal administration, or for administration by inhalation or insufflation, and may be formulated in an appropriate manner and in accordance with accepted practices such as those disclosed in Remington's Pharmaceutical Sciences, (Gennaro, ed., Mack Publishing Co., Easton Pa., 1990, herein incorporated by reference). The present invention also contemplates providing suitable topical formulations for administration to, e.g. mucosa.

For instance, for oral administration in the form of a tablet, the active drug component can be combined with an oral, non-toxic pharmaceutically acceptable inert carrier. Moreover, when desired or necessary, suitable binders, stabilizers, lubricants, disintegrating agents, flavoring agents and coloring agents can also be incorporated into the mixture. Suitable binders include, without limitation, starch, gelatin, natural sugars such as glucose or beta-lactose, natural and synthetic gums such as acacia, tragacanth or sodium alginate, carboxymethylcellulose, polyethylene glycol, waxes and the like. Lubricants used in these dosage forms include, without limitation, sodium oleate, sodium stearate, magnesium stearate, sodium benzoate, sodium acetate, sodium chloride and the like. Disintegrators include, without limitation, starch, methyl cellulose, agar, bentonite, xanthan gum and the like.

For preparing solid compositions such as tablets, the active ingredient is mixed with a suitable pharmaceutical excipient, and other pharmaceutical diluents, e.g. water, to form a solid preformulation composition containing a homogeneous mixture of a compound of the present invention, or a pharmaceutically acceptable salt thereof. By the term “homogeneous” is meant that the active ingredient is dispersed evenly throughout the composition so that the composition may be readily subdivided into equally effective unit dosage forms such as tablets, pills and capsules. The solid preformulation composition may then be subdivided into unit dosage forms of the type described above containing the active ingredient of the present invention.

The liquid forms in which the present compositions may be incorporated for administration orally include aqueous solutions, suitably flavored syrups, suspensions, and flavored emulsions with edible oils such as cottonseed oil, sesame oil, coconut oil or peanut oil, as well as elixirs and similar pharmaceutical carriers. Suitable dispersing or suspending agents for aqueous suspensions include synthetic and natural gums such as tragacanth, acacia, alginate, dextran, sodium carboxymethylcellulose, gelatin, methylcellulose or polyvinylpyrrolidone. Other dispersing agents which may be employed include glycerin and the like.

Consequently, the present invention also relates to a method of alleviating or treating a disease condition in which tolerance to autoantigen has a beneficial effect by administering a therapeutically effective amount of an autoantigen fusion polypeptide of the present invention to a subject in need of such treatment. Such diseases or conditions may, for instance arise from inappropriate immunological recognition of autoantigens. It is anticipated that by using the fusion polypeptides of the present invention, the problems with adverse side effects observed with the known therapies for autoimmune disease, such as steroids, may substantially be avoided.

The term “therapeutically effective amount” as used herein means that amount of active compound or pharmaceutical agent that elicits the biological or medicinal response in a tissue, system, animal or human that is being sought by a researcher, veterinarian, medical doctor or other clinician, which includes alleviation of the symptoms of the disease being treated.

Advantageously, autoantigen fusion proteins of the present invention may be administered in a single daily dose, or the total daily dosage may be administered in divided doses two, three or four times daily. Furthermore, autoantigen fusion proteins for the present invention may be administered in intranasal form via topical use of suitable intranasal vehicles well known to persons skilled in the art.

The dosage regimen utilizing the autoantigen fusion proteins of the present invention may be selected in accordance with a variety of factors including type, species, age, weight, sex and medical condition of the patient; the severity of the condition to be treated; the route of administration; the renal and hepatic function of the patient; and the particular compound employed. A physician or veterinarian of ordinary skill can readily determine and prescribe the effective amount of the drug required to prevent, counter or arrest the progress of the disease or disorder which is being treated.

The daily dosage of the products may be varied over a wide range from 0.01 to 100 mg per adult human per day. For oral administration, the compositions are preferably provided in pharmaceutical compositions containing, for examples, 0.01, 0.05, 0.1, 0.5, 1.0, 2.5, 5.0, 10.0, 15.0, 25.0 or 50.0 mg of the active ingredient for the symptomatic adjustment of the dosage to the patient to be treated. A unit dose typically contains from about 0.001 mg to about 50 mg of the active ingredient, preferably from about 1 mg to about 10 mg of active ingredient. An effective amount of the drug is ordinarily supplied at a dosage level of from about 0.0001 mg/kg to about 25 mg/kg of body weight per day. Preferably, the range is from about 0.001 to 10 mg/kg of body weight per day, and especially from about 0.001 mg/kg to 1 mg/kg of body weight per day.

Autoantigen fusion proteins according to the present invention may be used alone at appropriate dosages defined by routine testing in order to obtain optimal pharmacological effect. In addition, co-administration or sequential administration of other agents which improve the effect of the compound may, in some cases, be desirable.

The invention may be better understood by considering the following examples, which are provided to illustrate the invention but not to limit its scope. Other variants of the invention will be readily apparent to one of ordinary skill in the art and are encompassed by the appended claims.

### EXAMPLES

### Example 1

### Expression and Purification of CTB

Thirty cycles of polymerase chain reaction (each lasting 30 sec at 94° C., 30 sec at 60° C. and 2 min at 72° C.) were performed in a DNA thermal cycler (Perkin-Elmer Cetus Corp., Norwalk, Conn.) with V. cholerae 569 B chromosomal DNA using the following primers: Sense-5′-CTCCCATGGCTTTCGCTACACCTCAAAATATTACTG-3′[SEQ ID NO.39], the sequence encoding amino terminus portion of CTB fused to the sequence coding for carboxyl terminus of middle wall protein (MWP) signal peptide of B. brevis 47 that had a Nco I site; Antisense-5′-TGCAAGCTTACATGTTTGGGCAAAACGG-3′[SEQ ID NO.40], was the sequence complementary to that located 59 bp downstream from the CTB termination codon attached by a Hind III site sequence at its 5′ terminus. The resulting PCR products were cloned into a pT7Blue vector (Novagen Inc., Madison, Wis.). These plasmids were transformed into E. coli NovaBlue competent cells (Novagen). Bacteria were grown at 37° C. for 1 h and plated on LB plates containing 25 μg/ml of ampicillin, 35 μl of 25 mg/ml X-gal and 20 μl of 100 mM IPTG. Several strains carrying the CTB gene were grown at 37° C. for 16 h and plasmids were purified by the alkaline extraction method (Bimboim, HC (1983) Methods in Enzymol. 100: 243-255). After digestion with Nco I and Hind III, plasmids were fractionated on an agarose gel and then the 385 base pair of CTB fragment was purified by using GENECLEAN (Bio101, Vista, Calif.).

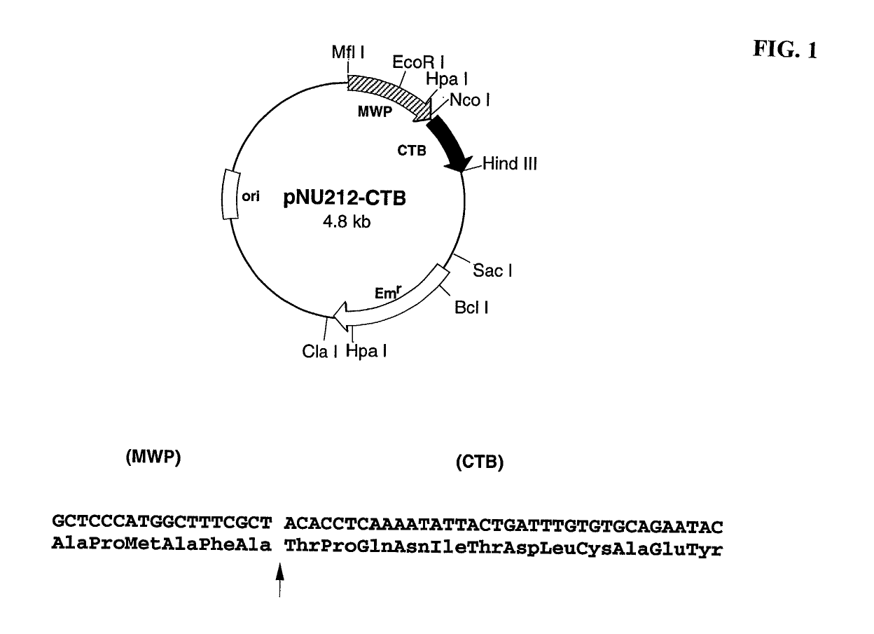

After ligation into pNU212, the plasmid DNA containing the CTB gene (pNU212-CTB, FIG. 1) was introduced into B. brevis 47K or HPD31 by the electroporation method (Okamoto, et al., (1997) Biosci. Biotech. Biochem. 61: 202-203) using the Gene Pulser II System (Bio-Rad Laboratories, Hercules, Calif.). B. brevis 47K and 31-OK carrying pNU212-CTB were grown for 3 days at 30° C. in S2U media containing 40 g of Soytone (Difco, Detroit, Mich.), 10 g of Yeast Extract (Difco), 30 g of glucose (Sigma Chemical Co. St. Louis, Mont.), 0.1 g of CaC122H20, 0.1 g of MgC127H20 and 0.1 g of Uracil (Sigma) per liter (pH 7.0) and in 5YC media containing 30 g of polypeptone (Difco), 20 g of Yeast Extract (Difco), 50 g of glucose (sigma), 0.1 g of CaC122H20, 0.1 g of MgC127H20, 0.01 g of FeSO47H20, 0.01 g of MnS044H20, 0.001 g of ZnS047H20, respectively. After the concentration of culture supernatants (1 L) with an Amicon (Beverly, Mass.) stirred cell and PM 10 membrane, the rCTB was precipitated with ammonium sulfate (80% saturation). The precipitate was dissolved in 50 mM Tris-HCl buffer (pH7.4) containing 0.2 M NaCl 3 mM NaN3, 1 mM EDTA (TEAN buffer) and the resulting solution was dialyzed against TEAN buffer. After centrifugation (20 min, 30,000 g), the dialysate was applied to a DEAE-Sepharose (Pharmacia Biotech, Alameda, Calif.) column (5×30 cm) equilibrated with TEAN buffer. The through-fraction was pooled and concentrated using the Amicon cell. After centrifugation (20 min, 30,000 g), the supernatant was applied to a galactose-immobilized gel (Pierce Chemical) column (2×15 cm) equilibrated with TEAN buffer. The column was washed with TEAN buffer and then eluted with 0.3 M galactose in TEAN buffer. The active fraction was pooled, concentrated by Amicon cell and then applied to a Sephadex G-100 (Pharmacia Biotech) column (2×95 cm) equilibrated with PBS, pH 7.4. Using this procedure, 250 mg of purified CTB was obtained from 1 L of B. brevis 47K culture supernatant. From 1 L of B. brevis 31-OK culture supernatant, 1000 mg of purified CTB was obtained.

### Example 2

### Expression and Purification of CTB-MBP 84-102

The open reading frame of a gene encoding CTB conjugated with MBP peptide was amplified by PCR followed by semi-nested PCR from the pNU212-CTB (FIG. 1) using the following primers:

| | | |
|---|---|---|
| Sense: | 5′-CTCCCATGGCTTTCGCTACACCTCAAAATATTACTG-3′,; | [SEQ ID NO.:41] |
| Antisense 1; | 5′-TTCTTGAAGAAGTGGACTACGGGGTTTTCATCATTGGCCATACTAAT-3′, | [SEQ ID NO.:42] |
| Antisense 2; | 5′-TAAGCTTAGGGTGGTGTGCGAGGCGTCACAATGTTCTTGAAGAAGTGGAC-3′. | [SEQ ID NO.:43] |

Thirty cycles of both polymerase chain reactions (each lasting 30 sec at 94° C., 30 sec at 60° C. and 2 min at 72° C.) were performed with a DNA thermal cycler (Perkin-Elmer Cetus Corp.). The resulting PCR products were cloned into a pT7Blue vector (Novagen Inc).

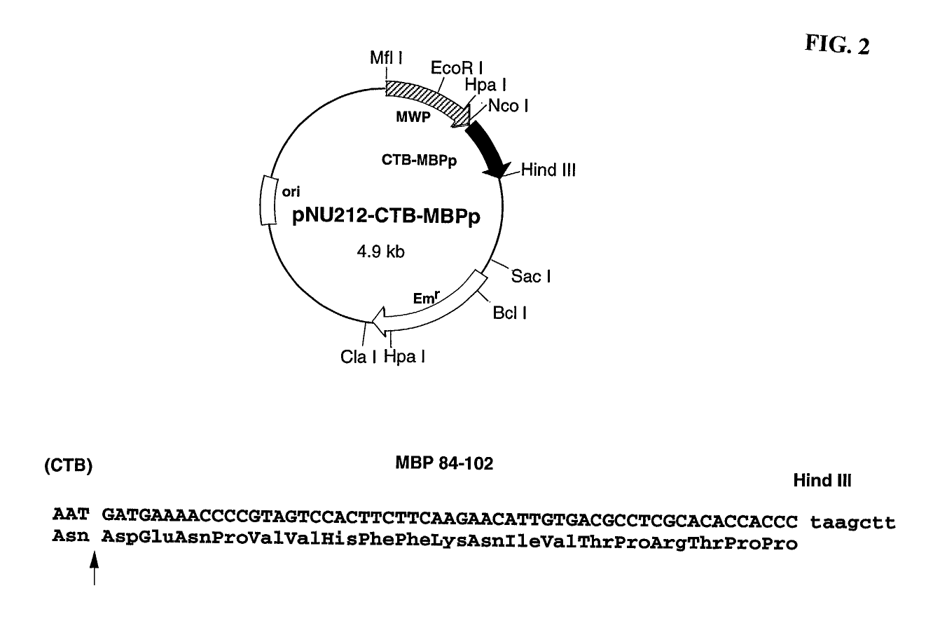

These plasmids were transformed into E. coli NovaBlue competent cells (Novagen). Bacteria were grown at 37° C. for 1 h and plated on LB plates containing 25 μg/ml of ampicillin, 35 μl of 25 mg/ml X-gal and 20 μl of 100 mM IPTG. Several strains carrying the CTB-MBP peptide gene were grown at 37° C. for 16 h and plasmids were purified by the alkaline extraction method. After digestion with Nco I and Hind III, plasmids were fractionated on an agarose gel and then the 381 base pair of CTB-MBP peptide fragment was purified by using GENECLEAN (Bio101). After ligation, the plasmid DNA containing the CTB-MBP peptide gene (pNU212-CTB-MBPp, FIG. 2) was introduced into B. brevis 47K or 31-OK by the electroporation method using the Gene Pulser II System (Bio-Rad Laboratories). After introduction of pNU212-CTB-MBPp into B. brevis 47K or HPD31, the clones producing rCTB-MBP peptide hybrid protein were identified in culture supernatants (47K in S2U media and 31-OK in 5YC media at 30° C.) by SDS-PAGE followed by Coomassie blue staining. Highly purified rCTB-MBP peptide hybrid proteins were obtained from 3-day culture supernatants of both B. brevis 47K and 31-OK by using ammonium sulfate precipitation, DEAE-Sepharose chromatography, galactose-immobilized affinity chromatography followed by gel filtration on a Sephadex G-100 column, as described above. Using this procedure, 60 mg of purified hybrid protein was obtained from 1 L of B. brevis 47K culture supernatant. From 1 L of B. brevis HPD31 culture supernatant, 250 mg of purified hybrid protein was obtained.

### Example 3

### Expression and Purification of CTB-PLP 13 9-151(C140S)

The open reading frame of a gene encoding CTB conjugated with PLP peptide was amplified by PCR from the pNU212-CTB using the following primers:

| | |
|---|---|
| Sense-5′-CTCCCATGGCTTTCGCTACACCTCAAAATATTACTG-3′, | [SEQ ID NO.:44] |
| Antisense-5′-AAGCTTAAAACTTGTCTGGATGTCCCAGCCATTTTCCCAAAGAATGATTGGCCATACT-3′. | [SEQ ID NO.:45] |

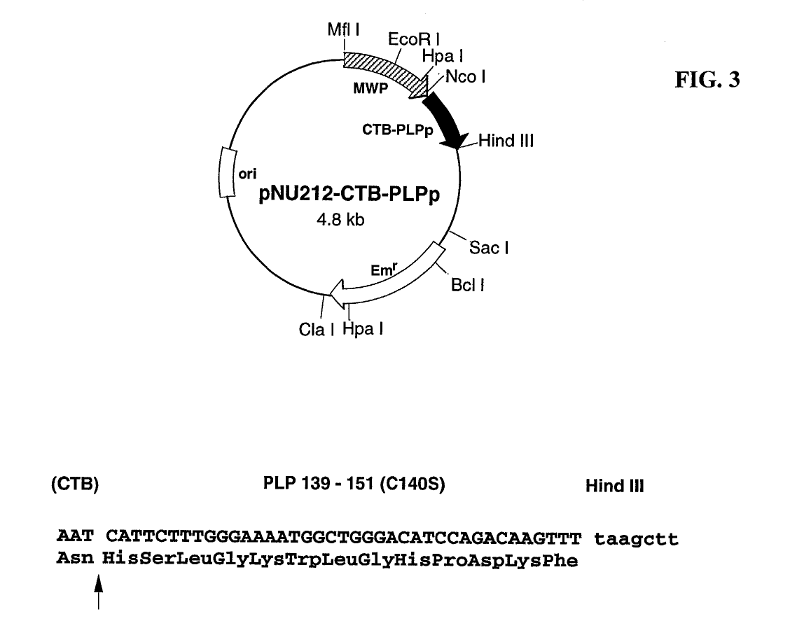

Thirty cycles of polymerase chain reaction (each lasting 30 sec at 94° C., 30 sec at 60° C. and 2 min at 72° C.) were performed with a DNA thermal cycler (Perkin-Elmer Cetus Corp.). The resulting PCR products were cloned into a pT7Blue vector (Novagen Inc). These plasmids were transformed into E. coli NovaBlue competent cells (Novagen). Bacteria were grown at 37° C. for 1 h and plated on LB plates containing 25 μg/ml of ampicillin, 35 μl of 25 mg/ml X-gal and 20 μl of 100 mM IPTG. Several strains carrying CTB-PLP peptide gene were grown at 37° C. for 16 h and plasmids were purified by the alkaline extraction method. After digestion with Nco I and Hind III, plasmids were fractionated on an agarose gel and then the 363 base pair of CTB-PLP peptide fragment was purified by using GENECLEAN (Bio101). After ligation, the plasmid DNA containing the CTB-PLP peptide gene (pNU212-CTB-PLPp, FIG. 3) was introduced into B. brevis 47K or 31-OK by the electroporation method using the Gene Pulser II System (Bio-Rad Laboratories). After introduction of pNU212-CTB-PLPp into B. brevis 47K or 31-OK, the clones producing rCTB-PLP peptide hybrid protein were identified in culture supernatants (47K in S2U media and HPD31 in 5YC media at 30° C.) by SDS-PAGE followed by Coomassie blue staining. Highly purified rCTB-PLP peptide hybrid proteins were obtained from 3-day culture supernatants of both B. brevis 47K and 31-OK by using ammonium sulfate precipitation, DEAF-Sepharose chromatography, galactose-immobilized affinity chromatography followed by gel filtration on a Sephadex G-100 column, respectively. Using this procedure, 100 mg of purified hybrid protein was obtained from 1 L of B. brevis 47K culture supernatant. From 1 L of B. brevis 31-OK culture supernatant, 500 mg of purified hybrid protein was obtained.

### Example 4

### Expression and Purification of CTB-PLP 139-11 (C140S) with a Hinge Peptide

The open reading frame of a gene encoding CTB conjugated with PLP peptide was amplified by PCR from the pNU212-CTB using the following primers:

| | |
|---|---|
| Sense-5′-CTCCCATGGCTTTCGCTACACCTCAAAATATTACTG-3′ and. | [SEQ ID NO.:46] |
| Antisense-5′-TAAGCTTAAAACTTGTCTGGATGTCCCAGCCATTTTCCCAAAGAATGgcctggaccATTGGCCATACT-3′. | [SEQ ID NO.:47] |

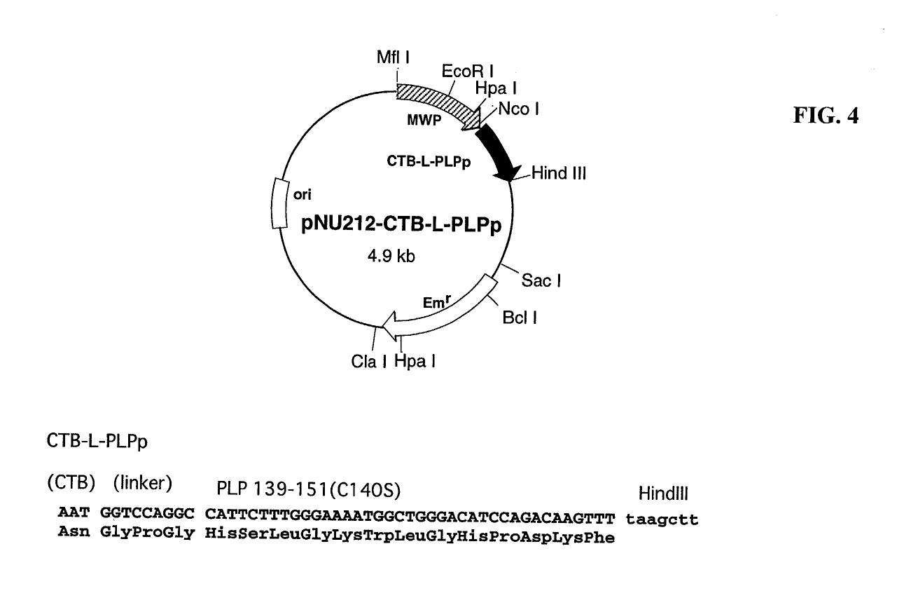

In this example, a DNA coding for Gly—Pro—Gly as a linker was inserted between that coding for CTB and PLP peptides. Twenty five cycles of both polymerase chain reactions (each lasting 30 sec at 94° C. 30 sec at 60° and 2 min at 72° C.) were performed with a DNA thermal cycler (Perkin-Elmer Cetus Corp.). The resulting PCR products were cloned into a pT7Blue vector (Novagen Inc.). These plasmids were transformed into E. coli NovaBlue competent cells (Novagen). Bacteria were grown at 37° C. for 1 h and plated on LB plates containing 25 μg/ml of ampicillin, 35 μl of 25 mg/ml X-gal and 20 μl of 100 mM IPTG. Several strains carrying CTB-GPG-PLP peptide were grown at 37° C. for 16 h and plasmids were purified by the alkaline extraction method. After digestion with Nco I and Hind III, plasmids were fractionated on an agarose gel and then the 372 base pair for CTB—GPG—PLP peptide fragment was purified by using GENECLEAN (Bio101). After ligation, the plasmid DNA containing CTB—GPG—PLP peptide gene (FIG. 4) was introduced into B. brevis 47K by the electroporation method using the Gene Pulser II System (Bio-Rad Laboratories). After introduction of pNU212-CTB-autoantigen peptide into B. brevis 47K, the clones producing CTB—GPG—PLP peptide hybrid protein was identified in culture S2U media producing CTB—GPG—PLP peptide hybrid protein was identified in culture S2U media supernatants by SDS-PAGE followed by Coomassie blue staining. Highly purified rCTB—GPG—PLP peptide hybrid protein was obtained from 3-day culture supernatants of B. brevis 47K by using ammonium sulfate precipitation, DEAE-Sepharose chromatography, galactose-immobilized affinity chromatography followed by gel filtration on a Sephadex G-100 column, as described above. Using this procedure, 70 mg of purified CTB-GPG-PLP peptide was obtained from 1 L of B. brevis 47K culture supernatant.

### Example 5

### Expression and Purification of CTB-Collagen Type II 255-270

The open reading frame of a gene encoding CTB fused to collagen Type II peptide was amplified by PCR from the pNU212-CTB using the following primers:

| | |
|---|---|
| Sense-5′-CTCCCATGGCTTTCGCTACACCTCA | [SEQ ID NO.:48] |
| AAATATTACTG-3′, | |

Antisense—CTB-Collagen type II 255-270

Antisense-CTB-Collagen type II 255-270

| | |
|---|---|
| 5′-CGTCGAAGCTTACTTTGGGCCTTGTTCACCT | [SEQ ID NO.:49] |
| TTGAAGCCAGCAATAGCAGGTTTACCCGTATTTG | |
| CCATACTAA-3′. | |

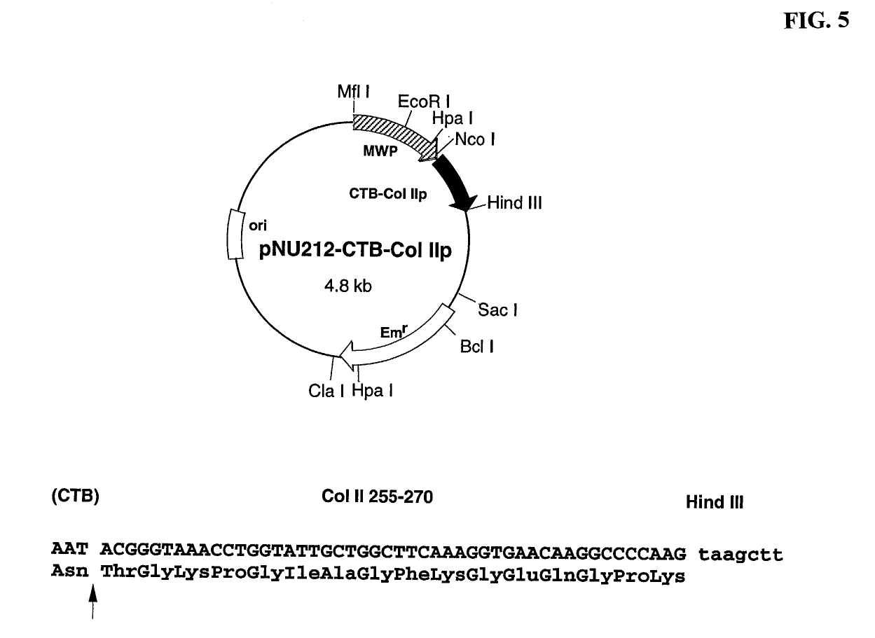

Twenty five cycles of polymerase chain reaction (each lasting 1 min at 94° C., 2 min at 57° C. and 1.5 min at 72° C.) were performed with a DNA thermal cycler (Perkin-Elmer Cetus Corp.). The resulting PCR products were cloned into a pT7Blue vector (Novagen Inc). These plasmids were transformed into E. coli NovaBlue competent cells (Novagen). Bacteria were grown at 37° C. for 1 h and plated on LB plates containing 25 μg/ml of ampicillin, 35 μl of 25 mg/ml X-gal and 20 μl of 100 mM IPTG. Several strains carrying CTB-collagen peptide gene were grown at 37° C. for 16 h and plasmids were purified by the alkaline extraction method. After digestion with Nco I and Hind III, plasmids were fractionated on an agarose gel and then the 372 base pair for CTB-Collagen Type II peptide were purified by using GENECLEAN (Bio101 ). After ligation, plasmid DNA containing CTB-collagen peptide gene (FIG. 5) was introduced into B. brevis 47K by the electroporation method using the Gene Pulser II System (Bio-Rad Laboratories). After introduction of pNU212-CTB-collage peptide into B. brevis 47K, the clones producing rCTB-auto collagen peptide hybrid protein was identified in culture S2U media supernatants by SDS-PAGE followed by Coomassie blue staining. Highly purified rCTB-autoantigen peptide hybrid proteins was obtained from 3-day culture supernatants of B. brevis 47K by using ammonium sulfate precipitation, DEAE-Sepharose chromatography, galactose-immobilized affinity chromatography followed by gel filtration on a Sephadex G-100 colunm. Using this procedure, 50 mg of purified CTB-collagen peptide hybrid protein was obtained from 1 L of B. brevis 47K culture supernatant.

### Example 6

### Amino Acid Sequence of the Hybrid Proteins

Purified CTB and typical two hybrid proteins were pyridylethylated with 4-vinylpyridine then separated on a reverse-phase HPLC column, Protein C4, (0.46×15 cm, Vydac, Hesperia, Calif.) (Fullmer, (1984) Anal. Biochem. 1142: 336-339), respectively. Each purified sample was dissolved in 0.1 M Tris-HCl (pH8.0) and digested with endopeptidase Lys-C (1:50 w/w, Wako, Chemicals, Richmond, Va.) at 37 C. Each digested sample was separated by reverse-phase HPLC on a C 18 HPLC column (0.46×15 cm, Vydac, Hesperia, Calif.) using a 0.1% trifluoroacetate (Buffer A)-80% acetonitrile in 0.1% trifluoroacetate (Buffer B) gradient system (Yuki et al., 1995). The analytical conditions were as follows: flow rate, 1.0 ml/min; detection, 220 nm; gradient program, 0-5 min (0% B), 5-20 min (0-20%), 20-45 min (20-35%, B), 45-65 min (35-65%, B), 65-70 min (65-100%, B). Each peak was collected, then analyzed by the Protein sequencer 610A (Perkin Elmer/Applied Biosystems, Foster City, Calif.). The N-terminal amino acid sequences of purified two hybrid proteins from both B. brevis 47K and HPD31, were identical to that of CTB. The amino acid sequencing also showed that hybrid protein rCTB-PLP 139-151 (C140S) peptides and rCTB-MBP 84-102 peptides from both B. brevis 47K and HPD31 contain the PLP 139-151(C140S) and MBP 84-102 amino acid sequence after the C-terminal of CTB, respectively, as shown below in Table 1. We also confirmed C-terminal amino acid as well as N-terminal one for other CTB-autoantigen peptide (Table 1).

TABLE 1
N-Terminal and C-terminal Sequences for CTB and the Chimera
Proteins.

| Sequence | Alignment |
|---|---|
| TPQNITDLCAEYHNTQIHTLNDK | 1-23 of CTB, CTB-MBP 84- |
| | 102 and CTP-PLP 139-151 |
| TPHAIAAISMAN | 92-103 of CTB |
| TPHAIAAISMANDENPVVHFFK | 92-113 of CTB-MBP 84-102 |
| NIVTPRTPP | 114-122 of CTB-MBP 84-102 |
| TPHAIAAISMANHSLGK | 92-108 of CTB-MBP 139-151 |
| | (C140S) |
| WLGHPDKF | 109-116 of CTB-PLP 139- |
| | 151 (C140S) |

### Example 7

### Cross-Linking SDS-PAGE

Purified typical rCTB-PLPp and rCTB-MBPp were individually cross-linked with dimethylpimelimidate (Pierce Chemical) using the method developed by Brew, et. al. ((1975) J. Biol. Chem. 250: 1434-1444). SDS-polyacrylamide gel electrophoresis (SDS-PAGE) was performed using the method described by Laemmli ((1970) Nature 22: 680-685) and the gel was stained with Coomassie brilliant blue R-250. The cross-linking analysis revealed these fusion proteins exist as pentamers since monomeric forms of these migrated at a molecular size of 11 Kda.

### Example 8

### Competitive Receptor ELISA

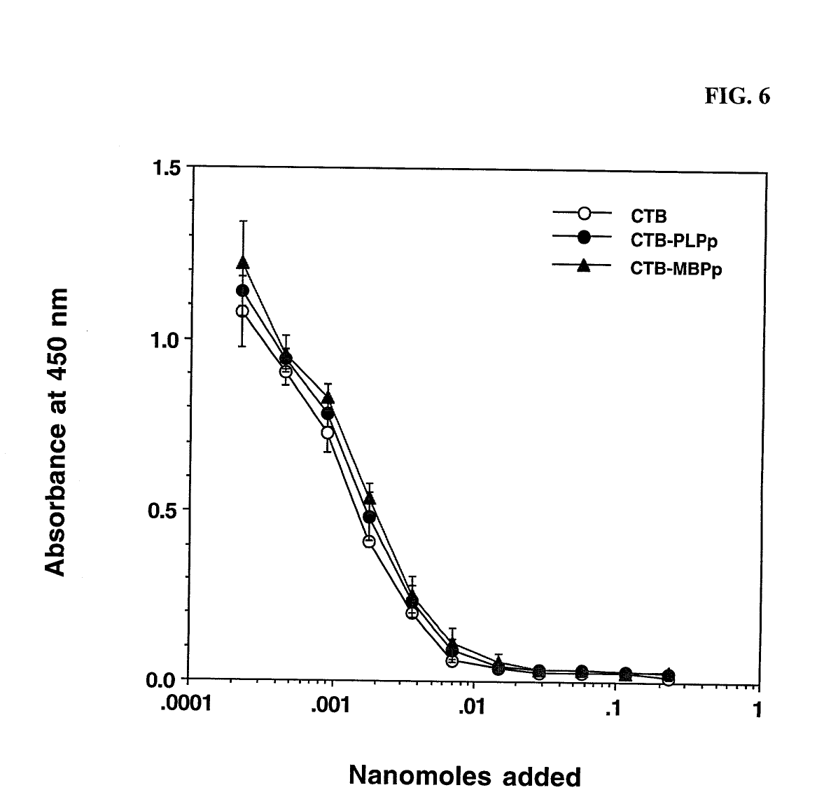

GM 1 receptor ELISA was carried out using the modified method of Dertzbaugh and Elson. ((1993)Infect. Immun. 42: 914-923). Wells of a polyvinyl microtiter plate were coated with 100 ng of GM1 ganglioside (Sigma). Wells were blocked with 1% bovine serum albumin in Tris-buffered saline, pH 7.5 (BSA-TBS). Each protein was adjusted to an equimolar concentration and then serially diluted two-fold in BSA-TBS. Each dilution was mixed with an equal volume of biotinylated CTB (List Biologics, Campbell, Calif.) diluted to a concentration of 100 ng/0.1 ml. After incubation for 2 h at room temperature; the plate was washed and horseradish peroxidase-conjugated streptavidin (Pierce Chemical) was added. The plate was incubated for 2 h at room temperature and, after washing, developed at room temperature with 100 g, 1 of chromogenic substrate, 3,3′, 5,5′-tetramethylbenzidine with H202 (Moss, Pasadena, Md.). Reactions were terminated by adding 50 μl of 0.5 M HCl. When increasing amounts of the rCTB-PLPp or rCTB-MBPp were mixed with a constant concentration of biotinylated CTB and then reacted with GM1, the rCTB-PLPp or rCTB-MBPp was found to bind to GM1 with an equivalent binding affinity as the native form of CTB (FIG. 6). The finding shows that the B. brevis-derived recombinant hybrid proteins, rCTB-PLPp and rCTB-MBPp possess a native pentamer form of CTB linked with PLP-139-151 (C140S) and MBP 84-102, respectively.

### Example 9

### Induction and Reduction of EAE

A peptide used in the induction of EAE, HSLGKWLGHPDKF [PLP 139-151(C140S); SEQ ID NO.:1], was chemically synthesized on a Applied Biosystems 430A peptide synthesizer (Foster City, Calif.). The peptide was more than 95% pure as determined by HPLC. Female SJL/J mice purchased from the Jackson Laboratory (Bar Harbor, Me.) were used at age 8-12 weeks. For induction of EAE (Elliott, et al. (1997) J. Neuroimmunol. 79: 1-11), SJL/J mice were primed subcutaneously with 100 nmol PLP 139-151(C140S) emulsified in complete Freund's adjuvant supplemented with 400 μg of Mycobacterium tuberculosis H37 RA (Difco Laboratories, Detroit, Mich.). On the day of and the day 2 after priming, each mouse was also injected intravenously with 0.2 μg of pertussis toxin (List Biologics, Campbell, Calif.). In order to assess the reduction of EAE, antigen-specific, mucosally induced tolerance was induced by nasal administration of 14-70 μg or oral administration of 175 μg of rCTB-PLPp on days 3, 4, 5, 6, 7 after priming with PLP139-151(C140S) and pertussis toxin for induction of EAE. Mice receiving PLP-peptide alone (10 or 100 μg) or PLP-peptide (10 μg) with CTB (80 μg) were used as a control group for those undergoing nasal immunization. Mice were routinely monitored for 40 days after systemic challenge with the PLP-peptide. The mice challenged with PLP-peptidel39-151(C140S) (control group) began to develop at around 6 days after the first PLP-peptide challenge, and the incidence of clinical disease reached 100% within approximately 12 days. When rCTB-PLPp was administered via the nasal or oral route, mice receiving 5 doses of 14 μg (Nasal 14 group) or of 70 μg (Nasal 70 group) or 175 μg (Oral 175 group) of the fusion protein showed a reduction in disease severity (Table 2; MCS=2.4±1.1, 2.0±0.7 and 2.4±12.0, respectively). The difference between Nasal 70 and control groups was statistically significant. Furthermore, incidence of recovery from paralysis in mice in the Nasal 70 group (9/10) was also higher than that of the control group (10/20) (Table 2). For comparison purposes, mice were nasally administered five times with 10 μg or 100 μg of PLP-peptide 139-151 (C140S) alone or with 10 μg of the PLP-peptide mixed with 80 μg of the rCTB. These two control nasal treatments did not provide the inhibitory effects necessary for reduction of the disease severity with paralysis as shown below in Table 2. These results show that the rCTB-PLPp is the most effective molecule for the inhibition of the EAE via the mucosal route.

TABLE 2
Effects of Nasal Treatment with rCTB-PLP Peptide Hybrid Protein for the Inhibition of EAE Development is SJL Mice.

| | | | | | Maximal | | |
|---|---|---|---|---|---|---|---|
| | | | Incidence of | | Clinical Score | | |
| Immunogen | Amount | Treatment | Paralysis | Mortality | (Mean) | Day of Onset (Mean) | Recovery |
| None | — | — (conrtol) | 20/20 | 2/20 | 3.2 ± 1.0 | 8.1 ± 2.2 | 10/20 |
| rCTB-PLP 139-151 | 14 μg | nasal (Nasal 14) | 9/10 | 0/10 | 2.4 ± 1.1 | 9.7 ± 1.8 | 7/10 |
| | 70 μg | nasal (Nasal 14) | 9/10 | 0/10 | 2.0 ± 0.7* | 8.7 ± 1.8 | 9/10 |
| | 175 μg | oral (Oral 175) | 10/10 | 0/10 | 2.4 ± 2.0 | 7.6 ± 2.0 | 7/10 |
| PLP 139-151 | 10 μg | nasal | 10/10 | 2/10 | 3.1 ± 0.9 | 7.9 ± 1.7 | 5/10 |
| | 100 μg | nasal | 10/10 | 1/10 | 3.3 ± 0.9 | 7.9 ± 1.5 | 5/10 |
| rCTP + PLP 139-151 | 80 μg + | nasal | 10/10 | 0/10 | 3.0 ± 0.9 | 8.3 ± 1.9 | 5/10 |
| | 10 μg | | | | | | |

The mean of the maximal clinical score and the mean day of onset represent the mean ± SD. Clinical severity was scored as follows: 0, no disease; 1, limp tail; 2, tail paralysis and hind limb weakness; 3, hind limb paralysis; 4, tetraplegia; 5, death.
p < 0.05 versus control group by Bonferroni's test.

### Example 10

### Measurement of DTH Responses

The immunogens were nasally administered on 3-7 days (5 times) after systemic challenge for the induction of EAE. Ten days after challenge with PLP139-151(C140S) for the induction of EAE, 30 μg of PLP139-151(C140S) in PBS was subcutaneously injected into the right hind footpad of both non-treated mice and those that had been nasally immunized with 70 μg of rCTB-PLPp, 100 μg of PLP peptide alone, or 10 μg of PLP peptide together with 80 μg of CTB. The left hind footpad received PBS as a control (Johnson, et al. (1998) Infect. Immun. 66:1666-1670). The PLP-peptide 139-151(C140S)-specific DTH responses were measured 10 days after challenge with PLP-peptide 139-151(C140S) for induction of EAE. Footpad thickness was measured before and 24 h after challenge. The differences in footpad swelling between the two footpads were taken as the DTH response. As shown in Table 3, DTH responses were reduced after 5 nasal administrations of 70 μg rCTB-PLPp subsequent to challenge with the PLP-peptide. On the other hand, control group receiving nasal treatment with the peptide, either alone or mixed with CTB, did not lead to the reduction of peptide specific DTH responses as shown below in Table 3). These findings demonstrate that tolerance can be nasally induced with the use of hybrid protein of CTB PLP-peptide alone.

TABLE 3
Nasal Immunization with rCTB-PLP Peptide Hybrid Protein Inhibits
EAE Peptide- Specific DTH Responses.

| | | Change in Foot Pad Thickness |
|---|---|---|
| Immunogen | Amount | (x 10³) |
| None (Control) | — | 40 ± 16 |
| RCTB-PLP 139-151 | 70 μg (Nasal 70) | 13 ± 9** |
| PLP 139-151 | 100 μg | 42 ± 16 |
| RCTB + PLP 139-151 | 80 μg + 10 μg | 38 ± 14 |

The differences in footpad swelling between the two footpads were taken as the DTH response.
** p < 0.01 versus control group by Dunnett's test.

### Example 11

### Histological Analyses

Mice previously tolerized or non-treated with rCTB-PLP peptide hybrid protein were sacrificed 10 days after challenge with the PLP peptide for induction of EAE. Spinal cords were removed and fixed in 10% phosphate-buffered formalin, and paraffin-embedded section were stained with luxol fast blue-hematoxylin and eosin for light microscopy examination. Histologic disease was quantified by counting the number of infiltrating perivascular/submeningeal cells (Kuchroo, et al., (1991) Pathobiology 59: 305-312). The leukocyte infiltration was scored as follows: grade 0, indicating the absence of cell infiltration; grade 1, infiltration in lone area; grade 2, infiltration in a few areas, grade 3, infiltration of a large number of cells in a few areas. The Wilcoxon's rank test was used for statistical analysis of the data. Histopathological studies of spinal cords from mice with nasally induced tolerance (Nasal 70 group) revealed less mononuclear leukocytes infiltration than in non-tolerized control mice. As shown in Table 4, incidence of perivascular/submeningeal infiltrations observed in specimens from control non-tolerized mice (2.4±05.) was significantly higher than that from mice nasally tolerized with rCTB PLP-peptide hybrid protein (1.7±0.8). These results suggest that there is a correlation between the induction of nasally induced tolerance for the inhibition of peptide-specific T cells and the reduction of lymphocyte migration to the spinal cord.

TABLE 4
Nasal Immunization with rCTB-PLP Peptide Hybrid Protein Reduces
Leukocyte Infiltration into CNS.

| | | Perivascular/submeningeal |
|---|---|---|
| Immunogens | Amount | cell infiltration |
| None (Control) | | 2.4 ± 0.5 |
| RCTB-PLP 139-151 | 70 μg (Nasal 70) | 1.7 ± 0.8* |
| The immunogens were nasally administered on 3-7 days (5 times) after systemic challenge for the induction of EAE. Ten days after the induction of EAE, all nasally treated mice (10 animals per group) were sacrificed and spinal cords were removed and then fixed in 10% phosphate-buffered formalin, and paraffin-embedded sections were stained with luxol fast blue-hematoxylin and eosin for light microscopy examination. Histologic disease was quantified by counting the number of | | |
| # infiltrating perivascular/submeningeal cells. The leukocyte infiltration was scored as follows: 0, indicating the absence of cell infiltration; 1, infiltration in lone area; 2, infiltration in a few areas; 3, infiltration of a large number of cells in a few areas. | | |

*p < 0.05 versus control group by Wilcoxon's rank test.

The invention described and claimed herein is not to be limited in scope by the specific embodiments herein disclosed, since these embodiments are intended as illustrations of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims.

The disclosures of all references cited herein are incorporated by reference in their entireties.

What is claimed:

1. An isolated polynucleotide encoding a fusion polypeptide comprising at least one proteolipid protein fragment fused in frame to a tolerogen polypeptide.

2. The polynucleotide of claim 1, wherein the proteolipid protein fragment comprises amino acids 139-151 of proteolipid protein as set forth in SEQ ID NO.:1

3. The polynucleotide of claim 1, wherein the proteolipid protein fragment is selected from the group consisting of: SEQ ID NO.:2; SEQ ID NO.:3; SEQ ID NO.:4; SEQ ID NO.:5; SEQ ID NO.:6; SEQ ID NO.:7; SEQ ID NO.:8; SEQ ID NO.:9; SEQ ID NO.:10; SEQ ID NO.:11; SEQ ID NO.:12; and SEQ ID 13.

4. The polynucleotide of claim 1, wherein the proteolipid protein fragment comprises a variant of SEQ ID NO.:1; SEQ ID NO.:2; SEQ ID NO.:3; SEQ ID NO.:4; SEQ ID NO.:5; SEQ ID NO.:6; SEQ ID NO.:7; SEQ ID NO.:8; SEQ ID NO.:9; SEQ ID NO.:10; SEQ ID NO.:11; SEQ ID NO.:12; or SEQ ID 13.

5. The polynucleotide of claim 4, wherein the proteolipid protein variant is a naturally occurring autoantigenic variant of proteolipid protein.

6. The polynucleotide of claim 4, wherein the proteolipid protein variant is a synthetic variant capable of inducing tolerance to proteolipid protein autoantigens.

7. The polynucleotide of claim 1, wherein the tolerogen polypeptide is cholera toxin B subunit.

8. The polynucleotide of claim 7, wherein the cholera toxin B subunits comprises the amino acid sequence set forth in SEQ ID NO.:14

9. The polynucleotide of claim 7, wherein the cholera toxin B subunit comprises a variant of the sequence set forth in SEQ ID NO.:14 capable of functioning as a tolerogen when fused to an autoantigen peptide.

10. The polynucleotide of claim 1, wherein the fusion polypeptide further comprises a flexible hinge polypeptide between one or more proteolipid protein fragment and the tolerogen polypeptide.

11. The polynucleotide of claim 10, where the flexible hinge polypeptide is selected from the group consisting of: SEQ ID NO.:15; SEQ ID NO.:16; SEQ ID NO.:17; SEQ ID NO.:18; and SEQ ID NO.:19.

12. An isolated polynucleotide encoding a polypeptide having the sequence set forth in SEQ ID NO:20.

13. An isolated polynucleotide having a sequence set forth in SEQ ID NO:21.

14. An isolated polynucleotide encoding a polypeptide having a sequence set forth in SEQ ID NO:22.

15. An isolated polynucleotide having a sequence set forth in SEQ ID NO:23.

16. An expression vector comprising the polynucleotide of claim 1 operatively linked to at least one transcriptional regulatory element.

17. A fusion polypeptide expressed from the expression vector of claim 16.

18. A pharmaceutical composition comprising the fusion polypeptide of claim 17 and a pharmaceutically acceptable carrier.

19. A host cell containing a vector of claim 16.

20. The host cell of claim 19, wherein the host is selected from the group consisting of bacteria, yeast, insect, plant and animal cells.

21. The host cell according to claim 19, wherein the host is a Bacillus species.

22. The host cell according to claim 19, wherein the host is Bacillus brevis.

23. A method for producing an autoantigen fusion protein comprising the steps of:
providing an expression vector, wherein a first polynucleotide sequence encoding an autoantigen is fused in-frame to a second polynucleotide sequence encoding a tolerogen, wherein the resulting fused polynucleotide sequences are operatively associated with regulatory sequences;
expressing autoantigen fusion polypeptide from the expression vector in Bacillus host cells; and
recovering the autoantigen fusion polypeptide.

24. The method of claim 23, wherein the first polynucleotide encodes a proteolipid protein fragment.

25. The method of claim 24, wherein the proteolipid protein fragment comprises amino acids 139-151 of proteolipid protein as set forth in SEQ ID NO.:1

26. The method of claim 24, wherein the proteolipid protein fragment is selected from the group consisting of:SEQ ID NO.:2; SEQ ID NO.:3; SEQ ID NO.:4; SEQ ID NO.:5; SEQ ID NO.:6; SEQ ID NO.:7; SEQ ID NO.:8; SEQ ID NO.:9; SEQ ID NO.:10; SEQ ID NO.:1; SEQ ID NO.:12; and SEQ ID NO.:13.

27. The method of claim 24, wherein the proteolipid protein fragment comprises a variant of SEQ ID NO.:1; SEQ ID NO.:2; SEQ ID NO.:3; SEQ ID NO.:4; SEQ ID NO.:5; SEQ ID NO.:6; SEQ ID NO.:7; SEQ ID NO.:8; SEQ ID NO.:9; SEQ ID NO.:10;SEQ ID NO.:11;SEQ ID NO.:12; or SEQ ID NO.:13.

28. The method of claim 27, wherein the proteolipid protein variant is a naturally occurring autoantigenic variant of proteolipid protein.

29. The polynucleotide of claim 27, wherein the proteolipid protein variant is a synthetic variant capable of inducing tolerance to proteolipid protein autoantigens.

30. The method of claim 23, wherein the first polynucleotide encodes a myelin oligodendrocyte glycoprotein fragment.

31. The method of claim 30, wherein the myelin oligodendrocyte glycoprotein fragment is selected from the group consisting of: SEQ ID NO.:24; SEQ ID NO.:25; and SEQ ID NO.:26.

32. The method of claim 23, wherein the myelin oligodendrocyte glycoprotein fragment comprises a variant of SEQ ID NO.:24; SEQ ID NO.:25; or SEQ ID NO.:26.

33. The method of claim 32, wherein the myelin oligodendrocyte glycoprotein variant is a naturally occurring autoantigenic variant of myelin oligodendrocyte glycoprotein.

34. The polynucleotide of claim 32, wherein the myelin oligodendrocyte glycoprotein variant is a synthetic variant capable of inducing tolerance to myelin oligodendrocyte glycoprotein autoantigens.

35. The method of claim 23, wherein the second polynucleotide encodes a cholera toxin B subunit.

36. The method of claim 36, wherein the cholera toxin B subunit comprises the amino acid sequence set forth in SEQ ID NO.:14.

37. The method of claim 36, wherein the cholera toxin B subunit comprises a variant of the sequence set forth in SEQ ID NO.:14 capable of functioning as a tolerogen when fused to an autoantigen peptide.

38. The method of claim 23, wherein the expression vector further comprises a third polynucleotide sequence encoding a flexible hinge polypeptide located between said first and second polynucleotides.

39. The method of claim 23, where the flexible hinge polypeptide is selected from the group consisting of: SEQ ID NO.:15; SEQ ID NO.:16; SEQ ID NO.:17; SEQ ID NO.:18; and SEQ ID NO.:19.

40. The method of claim 23, wherein said Bacillus host cells are Bacillus brevis cells.

41. An autoantigenic fusion polypeptide produced according to the method of claim 23.

42. A method of treating a neurodegenerative disease comprising the steps of:
providing an expression vector, wherein a first polynucleotide sequence encoding an autoantigen is fused in-frame to a second polynucleotide sequence encoding a tolerogen, wherein the resulting fused polynucleotide sequences are operatively associated with regulatory sequences;
expressing autoantigen fusion polypeptide from the expression vector in Bacillus host cells;
recovering the autoantigen fusion polypeptide; and
administering the autoantigen fusion polypeptide to a patient.

43. The method of claim 42, wherein the first polynucleotide encodes a proteolipid protein fragment.

44. The method of claim 44, wherein the proteolipid protein fragment comprises amino acids 139-151 of proteolipid protein as set forth in SEQ ID NO.:1.

45. The method of claim 44, wherein the proteolipid protein fragment is selected from the group consisting of: SEQ ID NO.:2; SEQ ID NO.:3; SEQ ID NO.:4; SEQ ID NO.:5; SEQ ID NO.:6; SEQ ID NO.:7; SEQ ID NO.:8; SEQ ID NO.:9; SEQ ID NO.:10; SEQ ID NO.:11; SEQ ID NO.:12; and SEQ ID 13.

46. The method of claim 44, wherein the proteolipid protein fragment comprises a variant of SEQ ID NO.:1; SEQ ID NO.:2; SEQ ID NO.:3; SEQ ID NO.:4; SEQ ID NO.:5; SEQ ID NO.:6; SEQ ID NO.:7; SEQ ID NO.:8; SEQ ID NO.:9; SEQ ID NO.:10; SEQ ID NO.:11; SEQ ID NO.:12; or SEQ ID 13.

47. The method of claim 46, wherein the proteolipid protein variant is a naturally occurring autoantigenic variant of proteolipid protein.

48. The method of claim 46, wherein the proteolipid protein variant is a synthetic variant capable of inducing tolerance to proteolipid protein autoantigens.

49. The method of claim 42, wherein the first polynucleotide encodes a myelin oligodendrocyte glycoprotein fragment.

50. The method of claim 49, wherein the myelin oligodendrocyte glycoprotein fragment is selected from the group consisting of: SEQ ID NO.:24; SEQ ID NO.:25; and SEQ ID NO.:26.

51. The method of claim 49, wherein the myelin oligodendrocyte glycoprotein fragment comprises a variant of SEQ ID NO.:24; SEQ ID NO.:25; or SEQ ID NO.:26.

52. The method of claim 51, wherein the proteolipid protein variant is a naturally occurring autoantigenic variant of proteolipid protein.

53. The method of claim 52, wherein the proteolipid protein variant is a synthetic variant capable of inducing tolerance to proteolipid protein autoantigens.

54. The method of claim 42, wherein the second polynucleotide encodes a cholera toxin B subunit.

55. The method of claim 54, wherein the cholera toxin B subunit comprises the amino acid sequence set forth in SEQ ID NO.:14.

56. The method of claim 54, wherein the cholera toxin B subunit comprises a variant of the sequence set forth in SEQ ID NO.:14 capable of functioning as a tolerogen when fused to an autoantigen peptide.

57. The method of claim 39, wherein the expression vector further comprises a third polynucleotide sequence encoding a flexible hinge polypeptide located between said first and second polynucleotides.

58. The method of claim 57, where the flexible hinge polypeptide is selected from the group consisting of: SEQ ID NO.:15; SEQ ID NO.:16; SEQ ID NO.:17; SEQ ID NO.:18; and SEQ ID NO.:19.

59. The method of claim 42, wherein the Bacillus host cells are Bacillus brevis cells

60. The method of claim 42, wherein the neurodegenerative disease is multiple sclerosis.

61. The method of claim 42, wherein the autoantigen fusion polypeptide is administered mucosally.

62. The method of claim 61, wherein the autoantigen fusion polypeptide is administered orally or nasally.

63. A method of ameliorating the symptoms of neurodegenerative disease comprising administering a therapeutically effective amount of the autoantigen fusion polypeptide of claim 41.

64. The method of claim 63, wherein the symptoms of neurodegenerative disease are selected from the group consisting of: loss of mobility, spasticity, pain, tremor, abnormal eye movements, paroxysmal symptoms, paralysis, bladder and bowel dysfunction, sexual disturbances, fatigue and depression.

65. A method of inducing tolerance to an autoantigen comprising administering an effective amount of pharmaceutical composition of claim 18.
